(19) United States
(12) Patent Application Publication
(10) Pub. No.: US 2013/0167848 A1
(43) Pub. Date: Jul. 4, 2013

(54) Heel Protection Device

(71) Applicants: Frank John Frassica, Pasadena, MD (US); Baris Sevinc, Huntington, CT (US); Jinjie Chen, Longmont, CO (US); Adam Christopher Diehl, Baltimore, MD (US); Yufeng Guo, Hershey, PA (US); Steve Shuo Wang, Baltimore, MD (US); Benjamin William Neese, Brentwood, TN (US); Stefan Ahldor Kowalski, Ashburn, VA (US)

(72) Inventors: Frank John Frassica, Pasadena, MD (US); Baris Sevinc, Huntington, CT (US); Jinjie Chen, Longmont, CO (US); Adam Christopher Diehl, Baltimore, MD (US); Yufeng Guo, Hershey, PA (US); Steve Shuo Wang, Baltimore, MD (US); Benjamin William Neese, Brentwood, TN (US); Stefan Ahldor Kowalski, Ashburn, VA (US)

(73) Assignee: THE JOHNS HOPKINS UNIVERSITY, Baltimore, MD (US)

(21) Appl. No.: 13/695,163

(22) PCT Filed: Apr. 27, 2011
(86) PCT No.: PCT/US2011/034058

§ 371 Date: Mar. 14, 2013

Related U.S. Application Data
(60) Provisional application No. 61/329,750, filed on Apr. 30, 2010.

(51) Int. Cl. A61F 15/00

(52) U.S. Cl. CPC A61F 15/008
USPC 128/888

(57) ABSTRACT

A heel protection method and device for the protection of heel ulcers includes an inflatable bladder adapted to be positioned adjacent a calf of a patient and a sensor adapted to be positioned adjacent a heel of the patient for detecting pressure at the heel. A microprocessor is adapted to receive a signal related to pressure at the heel, from which it determines whether an allowable level has been exceeded. If an excessive pressure level is sensed, the microprocessor transmits a signal to a separate device that alerts the caregiver to inflate the inflatable bladder or automatically by way of a pump operatively connected to the microprocessor. The method and device avoids excessive pressure at the heel by sensing the pressure at the heel, and redistributing the pressure from the heel to the calf if an allowable pressure level has been exceeded.

### REFERENCE TO RELATED APPLICATIONS

This application claims the benefit of U.S. Provisional Patent Application No. 61/329,750, filed on Apr. 30, 2010, which is hereby incorporated by reference for all purposes as if fully set forth herein.

### FIELD OF THE INVENTION

The present invention pertains to a heel protection device. More particularly, the present invention pertains to a heel protection device for prevention of heel ulcers.

### BACKGROUND OF THE INVENTION

Despite some improvement in reduction of pressure ulcers over the last decade, pressure ulcers remain a significant problem. Pressure ulcers form when pressure against the skin reduces blood supply to that area, causing the affected tissue to die. Pressure ulcers are particularly prevalent among long term care patients, who are confined to a bed or wheelchair for extended periods of time. However, pressure ulcers also occur over short periods of time, such as after an injury or surgical procedure.

Pressure ulcers can form in a number of different locations. One of the most common places for a pressure ulcer to form is on the heels of a patient. In particular, heel ulcers are the second most common form of pressure ulcers and account for 25% of all pressure ulcers. The incidence of pressure ulcers for patients in a hospital setting is around 7% and the prevalence of heel ulcers is around 17%. Thus, about 2 out of every 100 patients that enter the hospital will develop a heel ulcer. Even worse, the incidence of heel ulcers has been reported as being in a range of 19-32% in acute care facilities.

To address this concern, various products have been developed for the prevention of heel ulcers. For example, many health care facilities use various forms of heel protectors. One form of a heel protector is configured to be wrapped around the heel of the patient, and secured by way of a foam strap. This heel protector is made from resilient polyfiber, and includes ventilation holes to promote air circulation. However, this heel protector does little to reduce the pressure at the heel, and relies heavily upon a caregiver to shift the patient numerous times during the day. In fact, many of the products on the market fail to adequately relieve pressure at the heel over extended periods of time.

Accordingly, there is a need in the art for a heel protection device that prevents the formation of heel ulcers in both long-term and short-term patients.

### SUMMARY

According to a first aspect of the present invention, a heel protection device comprises a sock body configured for placement on a foot and calf of a user, the sock body including a heel portion for positioning about the heel of the user and a calf portion for positioning about the calf of the user, an inflatable bladder positioned adjacent the calf portion of the sock body for redistributing pressure to the calf from the heel, and a sensor positioned adjacent the heel portion of the sock body for detecting pressure in the heel.

According to a second aspect of the present invention, a method of protecting heel ulcers comprises placing a sensor beneath a heel of a subject, measuring the pressure beneath the heel by way of the sensor, and inflating an inflatable bladder adjacent to a calf of the subject when the measured pressure exceeds an allowable level.

According to a third aspect of the present invention, a heel protection device comprises an inflatable bladder adapted to be positioned adjacent a calf of a patient, a sensor adapted to be positioned adjacent a heel of the patient, the sensor for detecting pressure at the heel, and a microprocessor operatively connected to the sensor, and having a computer readable medium comprising software, wherein, wherein executed by the microprocessor, causes the microprocessor to receive a signal related to pressure at the heel, determine whether the allowable level has been exceeded, and transmit a signal to a pump to inflate the inflatable bladder or to a separate device to alert a caregiver that the inflatable bladder should be inflated.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a perspective view of an exemplary device according to the features of the present invention.

FIG. 2 illustrates a perspective view of an exemplary sock according to the features of the present invention.

FIG. 3 illustrates a perspective view of an exemplary inflatable bladder according to the features of the present invention.

FIG. 4 illustrates a top plan view of an exemplary inflatable bladder in a deflated condition according to the features of the present invention.

FIG. 5 illustrates a perspective view of an exemplary heel protection device according to the features of the present invention.

FIG. 6 illustrates a partial perspective view of an exemplary heel protection device according to the features of the present invention.

FIG. 7 illustrates a perspective view of an alternative device according to the features of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

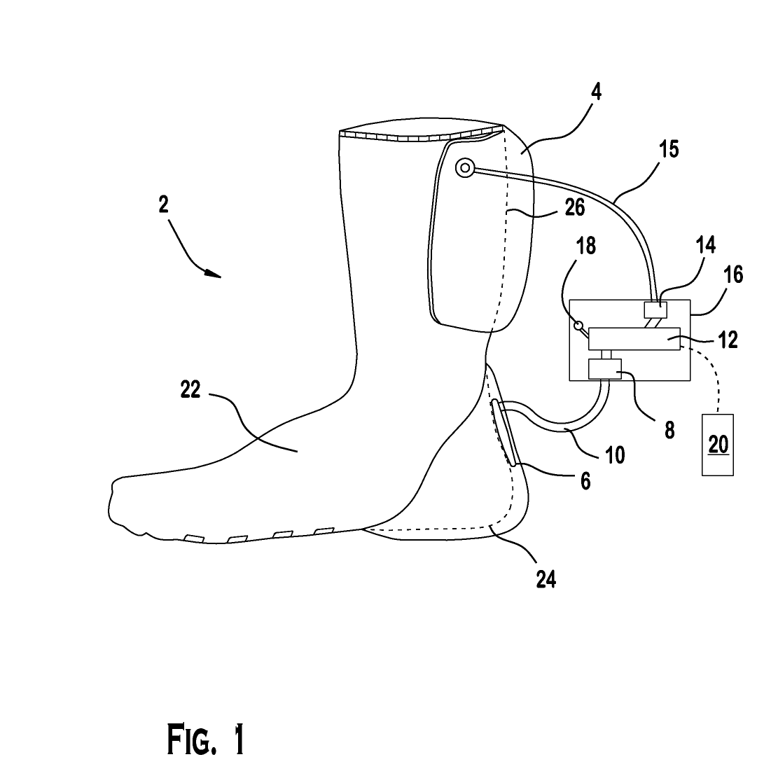

The present invention pertains to a heel protection device and method for the prevention of heel ulcers. With reference to FIG. 1, the heel protection device 2 includes an inflatable bladder 4 adapted for placement adjacent the calf of a patient. The inflatable bladder 4 is inflated when pressure at the heel exceeds an allowable level, so that pressure from the heel is redistributed to the calf. To detect pressure at the heel, a sensor 6 is adapted for placement adjacent the heel of the patient. The sensor 6 allows for pressure measurement at the heel, and provides information as to whether the pressure exceeds an acceptable level.

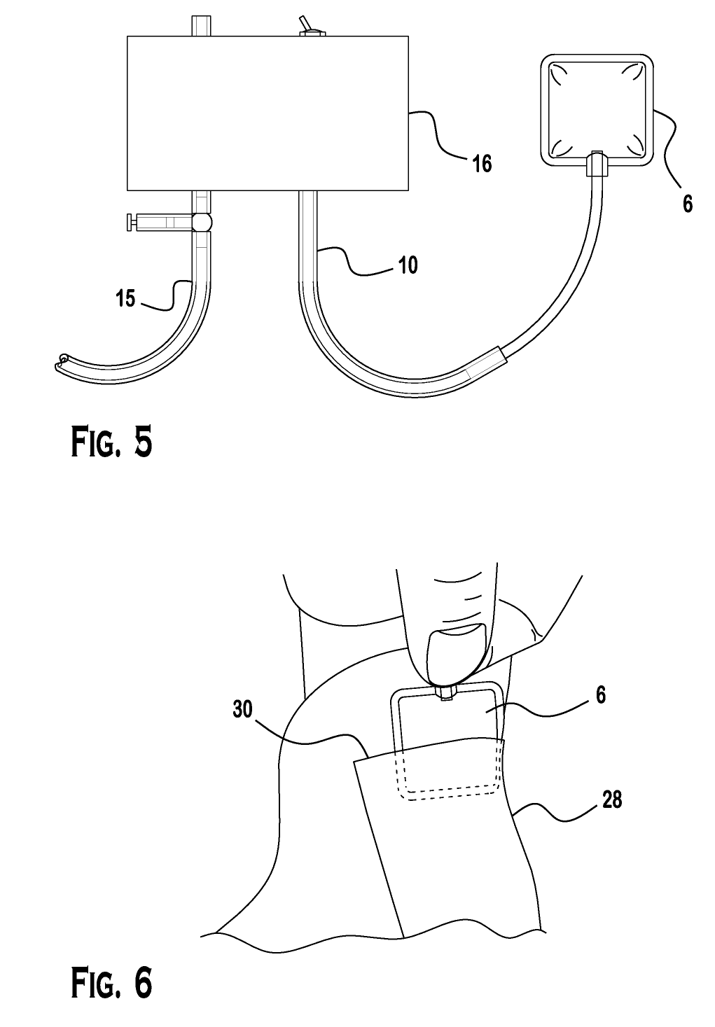

Preferably, the sensor 6 is a sensing bladder (see FIG. 5), and is operatively connected to a pneumatic pressure sensor 8 for determining pressure against the bladder 6 (see FIG. 1). For example, the sensing bladder may be connected to the pneumatic pressure sensor 8 via silicon tubing 10 or the like. The pneumatic pressure sensor 8 can measure the force applied to the sensing bladder 6, which is adjacent to the heel. However, it should be understood that any type of pressure sensor may be used, depending upon application and design preference.

With continued reference to FIG. 1, the sensor 8 is operatively connected to a microprocessor 12. The microprocessor 12 includes computer readable medium comprising software, wherein, wherein executed by the microprocessor 12, causes the microprocessor 12 to receive a signal related to measured pressure at the heel, and then determine whether an allowable level has been exceeded. Preferably, the allowable level should not exceed 17 mm Hg. However, this level will vary depending upon a patient's size, weight, and other factors, such as whether the patient is diabetic, has hypertension, or has poor circulation to the lower extremities. In addition, this level can be pre-programmed into the microprocessor 12, or set manually in the software.

Once the microprocessor 12 determines that an allowable pressure has been exceeded, the microprocessor 12 can transmit a signal to a pump 14 to inflate the inflatable bladder 4. Accordingly, the pump 14 is attached to the inflatable bladder 4 via silicon tubing 15, or the like. Preferably, the pump 14 is a micro air pump, and directs air to fill the inflatable bladder 4 at an appropriate level. However, it should be understood that pump 14 may be any device capable of inflating the inflatable bladder 4 with any type of medium.

Alternatively, the microprocessor 12 can send a signal to a separate device 20 to alert a caregiver that the inflatable bladder 4 should be inflated. Preferably, the separate device 20 is positioned at a nurse's station, or the like, to alert the caregiver that the inflatable bladder 4 needs inflation. The separate device 20 may include an LED, auditory alarm, or vibration mechanism. Upon receiving the signal, the caregiver can manually inflate the pump via a manual air pump or an electronic air pump. The separate device 20 may be any device separate from the heel protection device 2 that alerts a caregiver or the like that inflation of the device 2 is needed.

The microprocessor 12 may be programmed in a number of ways to maintain safety of the patient. For example, the microprocessor 12 may be programmed to inflate the inflatable bladder after a predetermined period of time. It may also include the ability to sense the pressure in the inflatable bladder, and to stop pumping if the pressure in the inflatable bladder reaches a dangerous level. In addition, the microprocessor 12 may be programmed such that intermittent pressure is applied to the calf, so that pressure is shifted between the calf and the heel. In this way, excessive pressure at the calf and heel is avoided. However, it should be understood that the microprocessor 12 may be programmed in numerous ways to avoid excessive pressure at the heel, depending upon application and design preference.

For ease of application, the pneumatic pressure sensor 8, microprocessor 12, and pump 14 may be incorporated into a small electronics housing box 16 (see also, FIG. 5). The electronics housing box 16 may optionally include fixation elements (such as VELCRO or the like), to attach the box 16 in a discrete location. In addition, the housing box 16 may include an indicator light 18 that operates when the allowable level has been exceeded. This will alert a caregiver when the allowable level has been exceeded.

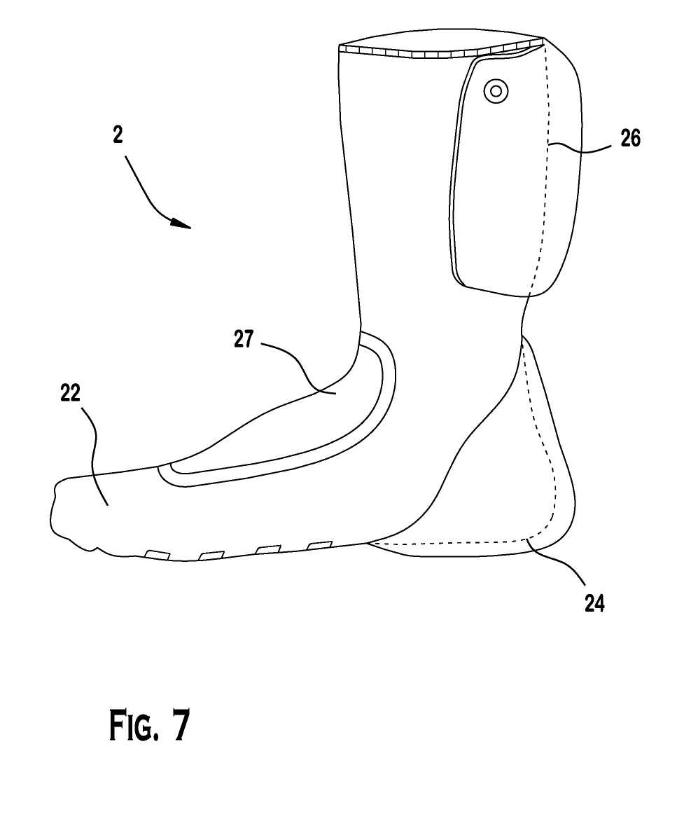

With reference to FIGS. 1, 2 and 7, the heel protection device 2 may include a sock body 22. The sock body 22 is configured for placement on a foot and calf of a user, and includes a heel portion 24 for positioning about the heel of the user and a calf portion 26 for positioning about the calf of the user. Preferably, the sock body is a compression sock, and is made from a moisture wicking fabric. Such fabrics include, but are not limited to, nylon, spandex, polyester, or a mixture thereof. The sock body 22 may also include an optional opening 27 at a top portion of the sock body 22 for allowing access to the heel (see FIG. 7).

The sock body 22 may also include a heel support 28 for protecting a heel of the user from friction and shear forces. Preferably, the heel support 28 is disposed adjacent to the heel portion 24 of said sock body 22. The heel support 28 is designed to provide extra padding at the area of the heel, and should be contoured to fit a heel of a user. Preferably, the heel support 28 is cup shaped, and is made from a gel-like material, such as Sorbothane, and the like. However, it should be understood that the heel support 28 may be made of other material for cushioning the heel, such as foam, air, liquid, or may also be in the form of an inflatable bladder. In addition, the heel support 28 may be removably attached to the sock body 22, or may be an integral part of the sock body, depending upon application and design preference.

With reference to FIG. 6, the heel support 28 includes a compartment 30 which allows a sensing bladder 6 to be placed therein. For example, the sock body 22 may be designed to include a pocket configured to receive the sensing bladder 6. Preferably, the compartment 30 is a fabric pocket, which allows the sensing bladder 6 to be positioned adjacent to the heel. However, it should be understood that a compartment 30 is not necessary, as the sensor 6 may be affixed to the heel in numerous ways, such as by tape, straps, or the like. In addition, the sensor 6 may be integrated into the sock body 22, or attached as a separate component, depending upon application and design preference.

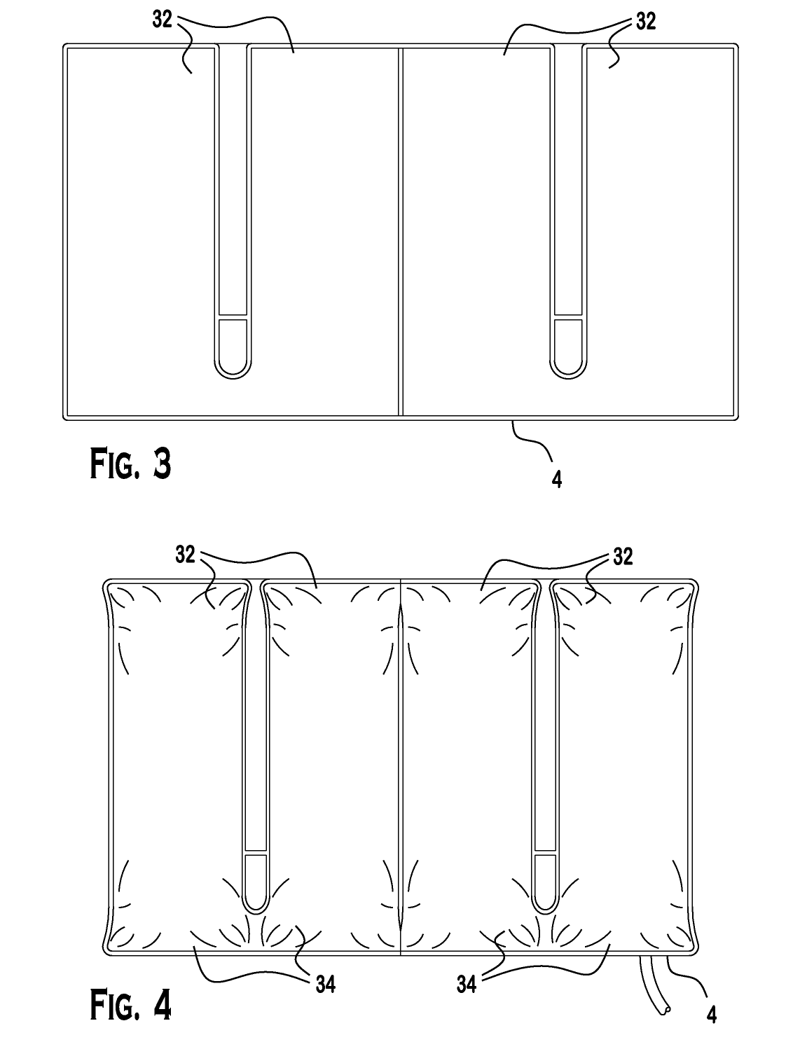

With reference to FIGS. 3 and 4, the inflatable bladder 4 will be described in more detail. Preferably, the inflatable bladder 4 includes a compartment that is inflatable such that the heel becomes elevated off the bed, thereby redistributing the pressure from the heel to the calf. Preferably, the inflatable bladder 4 includes a number of chambers 32 that more readily allow the inflatable bladder 4 to conform to the shape of the calf of a patient. In the exemplary embodiment, there are four chambers, wherein the inner two chambers are slightly larger and closer together than the outer two chambers. However, it should be understood that any number of chambers is possible, including just a single chamber, depending upon application and design preference. Moreover, while an air pump is described for filling the inflatable bladder 4 with air, any other type of inflation mechanism is possible, and within the scope of the present invention.

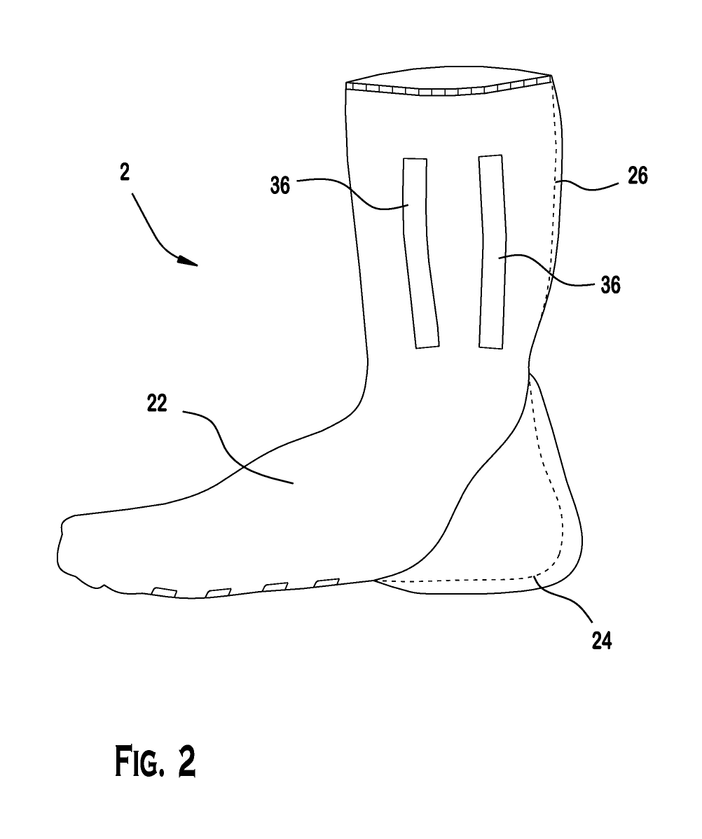

As shown with reference to FIGS. 2 and 4, the inflatable bladder 4 is preferably removably affixed to the calf portion 26 of the sock body 22. As illustrated in the exemplary embodiment, the inflatable bladder 4 includes VELCRO fastening elements 34 (FIG. 4), which are positioned for engagement with complimentary VELCRO fastening elements 36, positioned on the sock body 22 (FIG. 2). As shown in FIG. 4, there is one VELCRO strap on each chamber, which extends in the vertical direction. This allows the inflatable bladder 4 to be easily positioned in different locations along the calf of the patient, so that the optimal patient comfort is achieved. However, it should be understood that any type of detachable securing devices are possible, which can be placed in a number of different locations, depending upon application and design preference. In addition, no fastening elements are necessary on the inflatable bladder, as the inflatable bladder can be fixed to the patient in other ways, such as via tape or the like.

The inflatable bladder 4 should preferably be made from a material that can be sterilized and reused for another patient, and is preferably waterproof. Examples of such materials include, but are not limited to, vinyl, vinyl coated nylon, urethane, urethane coated nylon, ethyl vinyl acetate, polyurethane, PVC, and the like. In the exemplary embodiment, the inflatable bladder is made from polyurethane coated nylon. However, any type of material is possible, depending upon application and design preference.

It should be understood that the inflatable bladder 4 may be connected to a pre-existing stocking, such as a T.E.D.™ Anti-Embolism stocking used by a hospital, or the like. In this way, the material of the compression stocking may serve as a surface to receive the VELCRO fastening elements 34 of the inflatable bladder 4. However, as described above, the inflatable bladder 4 may be affixed in any other way to the pre-existing compression sock or sock body 22, such as by tape, straps, buttons, or the like.

### EXAMPLE

The following Example has been included to provide guidance to one of ordinary skill in the art for practicing representative embodiments of the presently disclosed subject matter. In light of the present disclosure and the general level of skill in the art, those of skill can appreciate that the following Example is intended to be exemplary only and that numerous changes, modifications, and alterations can be employed without departing from the scope of the presently disclosed subject matter. The following Example is offered by way of illustration and not by way of limitation.

### Example 1

The heel protection device is useful after a surgical procedure when a patient is recovering, and in the supine position for an extended period of time. Prior to the surgical procedure, compression stockings are placed onto the patient. An inflatable bladder (manufactured by Sealtech, located in Athens, Tenn.), is affixed to the compression stockings adjacent the calf of the patient. The inflatable bladder includes silicon tubing leading to a micro air pump (Model #JQB030-3A, distributed by Alibaba, located in Hong Kong), and is operatively connected to a microprocessor. A pressure sensor (HCX Series Fully Signal Conditioned Pressure Transducer, manufactured by SensorTechnics in Munich, Germany) is placed adjacent the heel of the patient, and operatively connected to the microprocessor.

A microprocessor, pressure transducer, and air pump are incorporated into an electronics box, which is affixed to the top portion of the compression stocking during use. The nurse turns on the device. The pressure sensor senses the pressure beneath the heel. When the pressure beneath the heel exceeds an allowable level, the inflatable bladder is inflated. The nurse can manually inflate the inflatable bladder via a hand pump, or allow the system to automatically fill the inflatable bladder with the requisite amount of air.

The heel protection device of the present invention is useful for many types of patients, such as those recovering in an acute care facility, as well as those in long term care facilities, such as nursing homes and the like. The heel protection device provides an automatic system for assuring that pressure at the heel is relieved over an extended period of time. It is also cost effective, as the electronics component as well as the inflatable bladder can be reused on other patients. The detachability of the inflatable bladder also allows the patient the ability to easily remove the device for increased mobility.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A heel protection device, comprising:
a sock body configured for placement on a foot and calf of a user, said sock body including a heel portion for positioning about a heel of the user and a calf portion for positioning about the calf of the user;
an inflatable bladder positioned adjacent the calf portion of the sock body for redistributing pressure to the calf from the heel; and
a sensor positioned adjacent the heel portion of the sock body for detecting pressure in the heel.

2. The heel protection device of claim 1, wherein the inflatable bladder is automatically inflated when said sensor detects a pressure above an allowable level.

3. The heel protection device of claim 2, wherein the allowable level is about 17 mmHg.

4. The heel protection device of claim 2, wherein said sensor sends a signal to a separate device for alerting a caregiver when the sensor detects a pressure above the allowable level.

5. The heel protection device of claim 1, wherein the sock body is made from a moisture-wicking fabric.

6. The heel protection device of claim 1, further including a heel support for protecting the heel of the user from friction and shear forces, said heel support being disposed in said heel portion of said sock body.

7. The heel protection device of claim 6, wherein the heel support is contoured to fit the heel of a user.

8. The heel protection device of claim 7, wherein the heel support is made from a gel or foam material.

9. The heel protection device of claim 6, wherein the heel support is an inflatable bladder.

10. The heel protection device of claim 1, wherein the sock body is in the form of a compression sleeve.

11. The heel protection device of claim 1, wherein said sock body includes an opening at a top foot portion of the sock body for allowing access to said heel.

12. The heel protection device of claim 1, wherein said inflatable bladder is manually operated.

13. The heel protection device of claim 1, wherein said inflatable bladder is removably affixed to said calf portion of said sock body.

14. The heel protection device of claim 2, further including a microprocessor having a computer readable medium comprising software, wherein, executed by the microprocessor, causes the microprocessor to receive a signal related to pressure at the heel, determine whether the allowable level has been exceeded, and transmit a signal to inflate the inflatable bladder.

15. The heel protection device of claim 14, wherein said microprocessor is operatively connected to an LED for alerting a caregiver when the allowable level has been exceeded.

16. The heel protection device of claim 14, further including a pump operatively connected to said microprocessor for automatically inflating said inflatable bladder when the allowable level has been exceeded.

17. The heel protection device of claim 14, further including a manual pump for inflating the inflatable bladder once the allowable level has been exceeded.

18. The heel protection device of claim 1, wherein the sensor is a sensing bladder disposed in a compartment of the heel support, said sensing bladder operatively connected to a pneumatic pressure sensor for determining pressure against the bladder.

19. A method of protecting a heel ulcer, comprising:
placing a sensor beneath a heel of a subject;
measuring the pressure beneath the heel by way of the sensor; and
inflating an inflatable bladder adjacent to a calf of the subject when the measured pressure exceeds an allowable level.

20. The method of claim 19, wherein the inflating step is performed automatically when the measured pressure exceeds the allowable level.

21. The method of claim 20, wherein a signal is sent to a separate device for alerting a caregiver when the allowable level has been exceeded.

22. A heel protection device, comprising:
an inflatable bladder adapted to be positioned adjacent a calf of a patient;
a sensor adapted to be positioned adjacent a heel of the patient, the sensor for detecting pressure at the heel;
a microprocessor operatively connected to said sensor, and having a computer readable medium comprising software, wherein, executed by the microprocessor, causes the microprocessor to receive a signal related to pressure at the heel, determine whether an allowable level has been exceeded, and transmit a signal to a pump to inflate the inflatable bladder or to a separate device to alert a caregiver that the inflatable bladder should be inflated.

23. The heel protection device of claim 22, wherein the inflatable bladder is automatically inflated when said sensor detects a pressure above the allowable level.

24. The heel protection device of claim 22, wherein the allowable level is about 17 mmHg.

25. The heel protection device of claim 22, wherein said sensor sends a signal to a separate device for alerting a caregiver when the sensor detects a pressure above the allowable level.

26. The heel protection device of claim 22, wherein said inflatable bladder is manually operated.

27. The heel protection device of claim 22, wherein said inflatable bladder is adapted to be removably affixed to a compression stocking.

28. The heel protection device of claim 22, wherein said microprocessor is operatively connected to an LED for alerting a caregiver when the allowable level has been exceeded.

29. The heel protection device of claim 22, further including a pump operatively connected to said microprocessor for automatically inflating said inflatable bladder when the allowable level has been exceeded.

30. The heel protection device of claim 22, further including a manual pump for inflating the inflatable bladder once the allowable level has been exceeded.

31. The heel protection device of claim 22, wherein said sensor is a sensing bladder operatively connected to a pneumatic pressure sensor for determining pressure applied against said sensing bladder.
